(19) United States
(12) Patent Application Publication
(10) Pub. No.: US 2023/0233887 A1
(43) Pub. Date: Jul. 27, 2023

(54) SELECTIVELY ACCESSIBLE MOUTH CLOSURE MASK

(71) Applicant: SNAP CPAP, LLC, Chapel Hill, NC (US)

(72) Inventor: Stuart Heatherington, Chapel Hill, NC (US)

(21) Appl. No.: 18/159,155

(22) Filed: Jan. 25, 2023

Related U.S. Application Data
(63) Continuation of application No. PCT/US2023/061207, filed on Jan. 25, 2023.
(60) Provisional application No. 63/302,741, filed on Jan. 25, 2022.

(51) Int. Cl. A62B 18/08; A62B 18/10; A62B 23/02

(52) U.S. Cl. CPC A62B 18/086; A62B 18/084; A62B 18/10; A62B 23/02; F16B 2001/0035

(57) ABSTRACT

A mouth closure mask includes a flexible sheet having an adhesive on a back facing surface thereof, the adhesive provided for adhering the mask to the face of a wearer. A port is defined through a portion of the sheet. The port allows access between in the inside and outside of a wearer's mouth when the port is uncovered. A retaining flange is disposed on the flexible sheet adjected to the port. A seal is configured to selectively engage the retaining flange to restrict access to the port when the seal and retaining flange are engaged.

### CROSS-REFERENCE TO RELATED APPLICATIONS

This application is a continuation of PCT Patent Application No. PCT/US2023/061207 entitled “SELECTIVELY ACCESSIBLE MOUTH CLOSURE MASK,” which was filed on Jan. 25, 2023, which claims benefit of and priority to U.S. Provisional Patent Application No. 62/302,741 which was filed on Jan. 25, 2022, the entire contents of which is hereby incorporated by reference in its entirety.

### TECHNICAL FIELD

The presently disclosed subject matter relates to a mouth closure mask, and particularly to a selectively accessible mouth closure mask for use over a wearer's mouth during sleep and that allows access to a wearer's mouth without removal of the mask.

### BACKGROUND

Snoring and obstructive sleep apnea (OSA) affects nearly 20% of all adults. Beyond causing discomfort for individuals and their sleeping partners, significant and even fatal medical consequences can and do occur because of these conditions.

These breathing difficulties can be caused by several different pressure points or resisters in the breathing process. Some of these resistors include the nose, the soft palate, the area behind the tongue, and the epiglottis. Any of these resistors can cause snoring, and certain individuals have multiple resistors simultaneously contributing to their breathing difficulties.

Snoring can further be rendered more likely or caused by a variety of factors which can include smoking, obesity, alcohol consumption, lack of exercise, and aging. All of these can have a negative influence on the general muscle tone of the upper airway, leading to an obstructed airway.

One approach to deal with snoring and OSA has been to seal over the mouth to force the wearer to breathe through their nose. This is typically accomplished with a pressure-sensitive adhesive mask that seals over the wearer's lips and/or portions of a wearer's face. The mask is largely a single use mask. Accordingly, if the wearer wakes in the middle of the night to consume liquids or to talk to someone, they often will have to remove the mask and then reapply another separate mask.

Accordingly, it would be beneficial to provide an improvement that addresses the above-noted disadvantages, including allowing a wearer to consume liquids or talk to someone without removing a mask.

### SUMMARY

This summary is provided to introduce in a simplified form concepts that are further described in the following detailed descriptions. This summary is not intended to identify key features or essential features of the claimed subject matter, nor is it to be construed as limiting the scope of the claimed subject matter.

Provided herein is a mouth closure mask that is selectively accessible to allow a wearer to consume liquids or talk without removal thereof. According to various embodiments, the mouth closure mask comprises a flexible sheet having an adhesive on a back facing surface thereof with the adhesive configured for adhering the mask to the face of a wearer. The flexible sheet further comprises a port defined through a portion of the sheet allowing access between the inside and outside of a wearer's mouth when the port is uncovered. The mouth closure mask also comprises a retaining flange disposed on the flexible sheet adjacent to the port. The mouth closure mask further comprises a seal configured to selectively engage the retaining flange restricting access to the port when the seal and retaining flange are engaged.

According to one or more embodiments, the retaining flange is disposed on a portion of a front facing surface of the flexible sheet.

According to one or more embodiments, the retaining flange and the seal are magnetically attracted to allow selective engagement. According to further embodiments, the seal that is magnetically attracted to the retaining flange includes a magnet assembly comprising a plurality of magnets mounted within the seal. In additional such embodiments, each of the magnets have a portion configured to magnetically engage the retaining flange and disposed such that each of the plurality of magnets has a polarity that is opposed to that of adjacently disposed magnets mounted within the seal. According to further embodiments, the retaining flange that is magnetically attracted to the seal includes a magnet assembly comprising a plurality of magnets mounted within the retaining flange. In additional such embodiments, each of the magnets have a portion configured to magnetically engage the seal and disposed such that each of the plurality of magnets has a polarity that is opposed to that of adjacently disposed magnets mounted within the retaining flange.

According to one or more embodiments, the seal includes a magnet assembly and the retaining flange includes a ferrous ring.

According to one or more embodiments, the seal includes a ferrous material and the retaining flange includes a magnet assembly.

According to one or more embodiments, the seal and the retaining flange include attractive magnet assemblies.

According to one or more embodiments, the seal and the retaining flange are selectively operatively coupled.

According to one or more embodiments, the seal and retaining flange are selectively operatively coupled with a hook and loop fastener assembly.

According to one or more embodiments, the seal comprises an enclosure defining a passageway that is in fluid communication with the port.

According to one or more embodiments, the seal comprises a one-way valve, or a valve that is configured to restrict flowthrough more in one direction than an opposed direction.

According to one or more embodiments, the seal comprises an insert configured to be received within the passageway to restrict access through the passageway and the port.

According to one or more embodiments, the insert carries a mesh or filter material.

According to one or more embodiments, the seal includes a tab for a wearer to selectively disengage the seal from the retaining flange.

According to various embodiments, the mouth closure mask comprises a flexible sheet having a front-facing surface and an opposed back-facing surface with adhesive on the back-facing surface. The flexible sheet further comprises a loop-type fabric portion of a hook and loop fastener assembly disposed on the front facing surface. The flexible sheet further comprises a port defined through a portion of the sheet allowing access between the inside and outside of a patent's mouth when the port is uncovered. The mouth closure mask further comprises a seal that comprising a hook-type fabric portion of a hook and loop fastener assembly, the hook-type fabric portion of the seal configured to selectively engage the loop-type fabric portion so the seal covers the port restricting access therethrough.

According to one or more embodiments, the seal comprises an enclosure defining a passageway that is in fluid communication with the port.

According to one or more embodiments, the seal comprises a one-way valve, or a valve that is configured to restrict flowthrough more in one direction than an opposed direction.

According to one or more embodiments, the seal comprises an insert configured to be received within the passageway to restrict access through the passageway and the port.

According to one or more embodiments, the hook-type fabric portion of the seal fully covers the port preventing access therethrough.

### BRIEF DESCRIPTION OF THE DRAWINGS

FIG. 1 illustrates a side view of a mouth closure mask for treating snoring and sleep apnea according to one or more embodiments disclosed herein;

FIG. 2 illustrates a top facing, perspective view of a mouth closure mask for treating snoring and sleep apnea according to one or more embodiments disclosed herein;

FIG. 3 illustrates a bottom facing, perspective view of a mouth closure mask for treating snoring and sleep apnea according to one or more embodiments disclosed herein;

FIG. 4 illustrates a side view of a mouth closure mask according to the embodiment of FIG. 1 with the seal selectively disengaged therefrom;

FIG. 5 illustrates a side view of a mouth closure mask according to one or more embodiments disclosed herein having the seal disengaged so a wearer wearing the mask is able to drink through a straw;

FIG. 6 illustrates an top facing, perspective exploded view of the mask according to one or more embodiments disclosed herein with the enclosure being transparent to show individual magnets therein and with a seal including an enclosure having a valve and tab;

FIG. 7A illustrates a side view of a mouth closure mask according to one or more embodiments disclosed herein having a washer-shaped ferrous assembly retaining flange and washer-shaped magnet assembly bottom portion of the enclosure which are magnetically attractive;

FIG. 7B illustrates a side view of a mouth closure mask according to one or more embodiments disclosed herein having a washer-shaped magnet assembly retaining flange and washer-shaped ferrous assembly bottom portion of the enclosure which are magnetically attractive;

FIG. 7C illustrates a side view of a mouth closure mask according to one or more embodiments disclosed herein having a washer-shaped retaining flange and washer-shaped bottom portion of the enclosure which are magnetically both magnet assemblies and magnetically attractive;

FIG. 8 illustrates a side view of a mouth closure mask according to one or more embodiments disclosed herein having a loop type portion of a hook and loop fastener affixed to a front facing side of the flexible sheet and a hook type portion of a hook and loop fastener as a seal that selectively engages the loop type portion to restrict access to the port; and

FIG. 9 illustrates a side view of a mouth closure mask according to one or more embodiments disclosed herein having washer shaped hook and loop fastener portions affixed to the flexible sheet and the enclosure of the seal.

### DETAILED DESCRIPTION OF THE EMBODIMENTS

Below, the technical solutions in the examples of the present invention are depicted clearly and comprehensively with reference to the figures according to the examples of the present invention. Obviously, the examples depicted here are merely some examples, but not all examples of the present invention. In general, the components in the examples of the present invention depicted and shown in the figures herein can be arranged and designed according to different configurations. Thus, detailed description of the examples of the present invention provided in the figures below are not intended to limit the scope of the present invention as claimed, but merely represent selected examples of the present invention. On the basis of the examples of the present invention, all of other examples that could be obtained by a person skilled in the art without using inventive efforts will fall within the scope of protection of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

These and other changes can be made to the disclosure in light of the Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description of The Embodiments section explicitly defines such terms. Accordingly, the actual scope of the disclosure encompasses not only the disclosed embodiments, but also all equivalent ways of practicing or implementing the disclosure under the claims.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments+/−20%, in some embodiments+/−10%, in some embodiments+/−5%, in some embodiments+/−1%, in some embodiments+/−0.5%, and in some embodiments+/−0.1%, from the specified amount, as such variations are appropriate in the disclosed packages and methods.

Various embodiments of the presently disclosed subject matter are directed to a facially applied mouth closure mask assembly 10 (hereafter identified as “mask”) that can allow access to the wearer's mouth through a port 16 without removal of the mask 10 itself. Due to the use of adhesive 14 to apply the mask 10 and characteristics of most adhesives 14, removal of a mask generally degrades the quality of the adhesive and its ability to securely adhere to a wearer's face around their mouth. Indeed, removal of most mouth closure masks generally necessitate the reapplication of a separate new one.

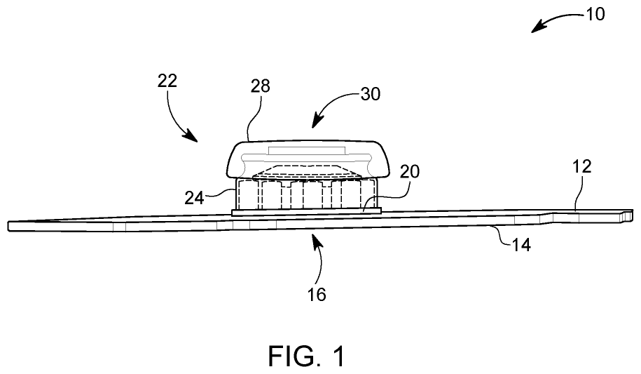

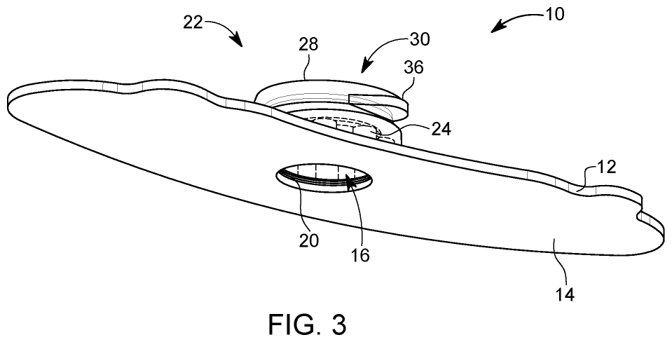

Various embodiments, including those illustrated in the accompanying figures are directed to a mask 10 that acts as a sleep aide by helping to alleviate breathing problems associated during sleep, such as snoring and obstructive sleep apnea (OSA). In various embodiments, the mask 10 includes a flexible sheet 12 having a front facing surface and an opposed back facing surface with adhesive 14 thereupon, as shown in FIGS. 1 and 3. In such embodiments, the adhesive 14 is provided for adhering the mask 10 to the face of a wearer. In embodiments, the adhesive 14 is pressure sensitive and will generally have just enough “tack” to adhere to the wearer while still allowing painless removal of the mask 10 when the wearer is done using the mask 10, thereby making the mask 10 disposable and for a single use. In various embodiments, the sheet can be made of any suitable material including, as non-limiting examples, cloth, woven and non-woven fabric, paper, gauze, and polymers such as polypropylene, polyurethane, polyacrylonitrile, polystyrene, polycarbonate, polyethylene, and polyester. Moreover, in certain embodiments, it is foreseen that the mask 10 can be configured for more than a single application and removal, such as by varying the adhesive 14. Indeed, in variations on a multiple use embodiment, the mask 10 also includes a removably attached protector to cover the adhesive 14 or a case to allow the mask 10 to be stored. In at least one embodiment, the mask 10 includes a separate new adhesive layer that a wearer affixes to the sheet 12, covering the pre-utilized adhesive 14, which includes new unused adhesive to secure the mask 10, now including the sheet 12 and new adhesive layer, to a wearer.

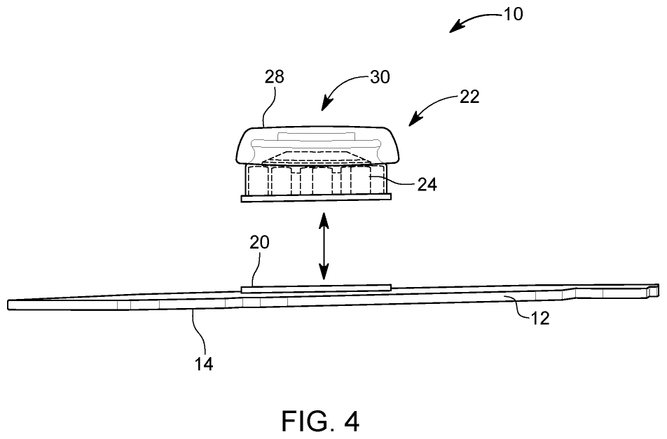

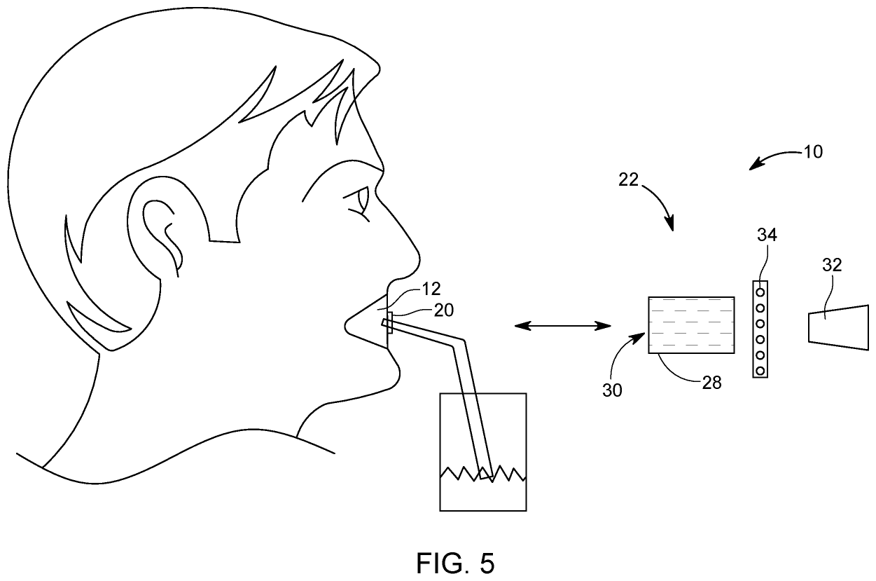

In further embodiments, the mask 10 will include a port 16, as identified in FIGS. 1 and 3, defined through a portion of the sheet 12 to allow access between the outside and inside of a wearer's mouth when the mask 10 is applied. In embodiments, the port 16 is a circular cut out, or hole. However, it is foreseen that any desired or appropriate shape may be employed for the port 16. As shown in FIG. 5, the port 16 is configured allow access between the inside and outside of a wearer's mouth when the port 16 is uncovered allowing a wearer to talk and/or drink without removal of the mask 10. In certain embodiments, the mask 10 may also include a retaining flange 20 carried by the flexible sheet 12 about the port 16, as indicated in FIGS. 1, 3, 4, and 6. The retaining flange 20 will be described in more detail to follow.

In various embodiments, a seal 22 is configured to selectively engage the retaining flange 20 to thereby restrict access to the port 16 when the seal and retaining flange are engaged. Therefore, the seal 22 restricts flow-through of air or liquids. In embodiments, the seal 22 is a cylindrical enclosure, as shown in FIGS. 1-6. However, it is foreseen that any desired or appropriate configured shape may be employed. In certain embodiments, the retaining flange 20 and the seal 22 are magnetically attracted to allow selective engagement, as in FIGS. 1-4, 6, and 7. Accordingly, seal 22 can be selectively engaged with the mask 10 to prevent access between the inside and outside of a wearer's mouth while the wearer is sleeping. Moreover, seal 22 may be selectively removed from the mask 10, allowing access between the inside and outside of a wearer's mouth without removal of the mask 10. Due to the selective engagement of the seal 22, a wearer would not have to remove the mask 10 to drink liquids through a straw or talk, as shown in FIG. 5.

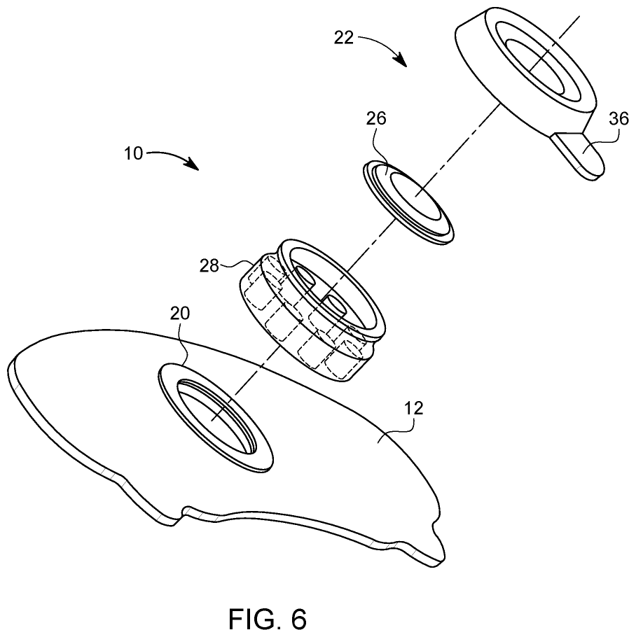

In certain embodiments, the seal 22 comprises a magnet assembly having a plurality of individual magnets 24 mounted therein and the retaining flange 20 comprises a magnetically attractive substance (such as a ferrous metal), as shown in FIG. 1. Indeed, in one embodiment, the seal 22 can comprise eight (8) individual magnets 24, as shown in FIG. 6. Moreover, in embodiments, the individual magnets 24 might have a portion, such as an end thereof, configured to engage the retaining flange 20. In certain embodiments, the polarity of the end portion of the individual magnets 24 configured to allow magnetic attraction and engagement have a polarity that is opposed to that of neighboring or adjacent individual magnet end portions. Thereby, the magnet assembly of the seal 22 might more securely affix to the magnetically attractive retaining flange 20.

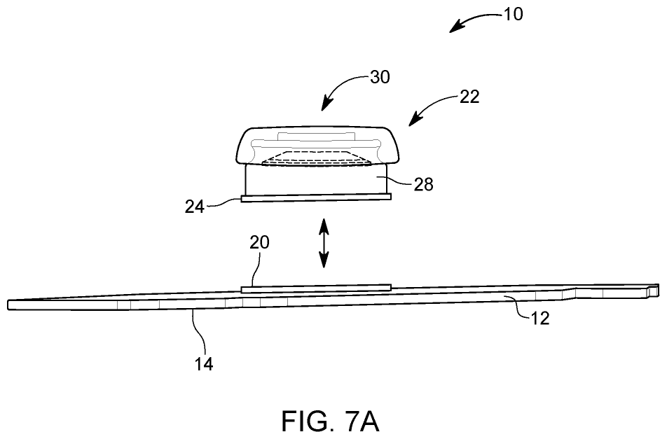

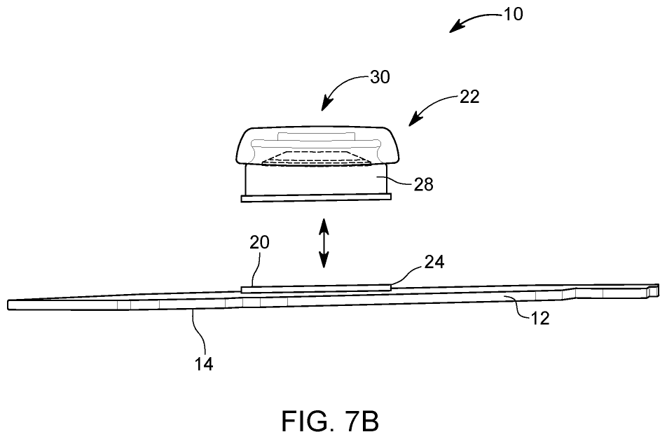

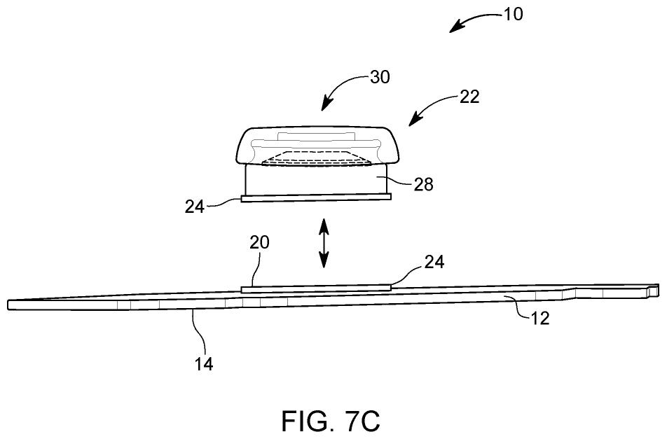

While the above and the figures recite a particular number and configuration of individual magnets, it is foreseen that any desired or appropriate number or configuration may be used. Indeed, in certain embodiments, one or both of the seal 22 and retaining flange may be a magnet assembly having a single magnet shaped as a closed disc, like a washer as in FIGS. 7A-7C. Moreover, while the above provides embodiments where the seal 22 comprises a magnet assembly and the retaining flange 20 comprises a ferrous assembly (such as in FIG. 7A), in other embodiments the retaining flange 20 can be a magnet assembly and the seal 22 can be a ferrous assembly as in FIG. 7B. Further, in other embodiments, both the retaining flange 20 and seal 22 can comprise attractive magnet assemblies as in FIG. 7C. Indeed, in at least one embodiment, both the retaining flange 20 and the seal 22 might comprise closed disc, washer shaped, magnets which are attractive to each other as in FIG. 7C.

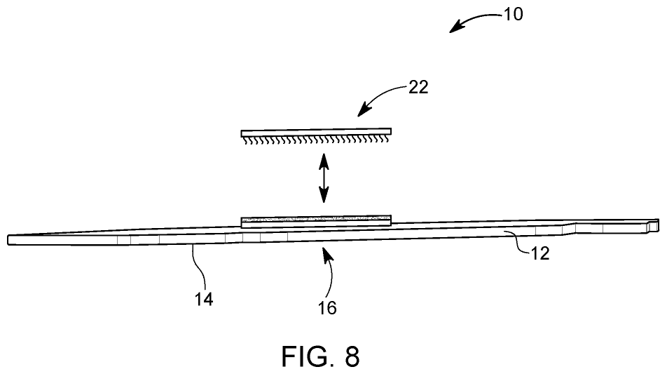

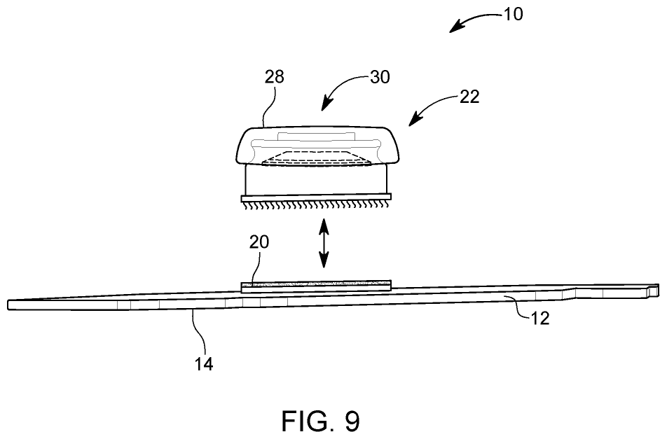

In other embodiments, retaining flange 20 and seal 22 can be operatively coupled through other fastener options, such as a hook and loop fastener assembly as in FIGS. 8 and 9. Indeed, in at least one embodiment, retaining flange 20 might have a loop-type fabric portion of a hook and loop fastener assembly and the seal 22 might have a hook-type fabric portion of a hook and loop fastener assembly disposed upon a portion thereof and configured to engage the loop-type fabric portion of the flange as in FIG. 9. Further, in other embodiments, the front-facing surface of the sheet 12 might have a loop-type fabric portion of a hook and loop fastener assembly disposed thereupon and the seal 22 itself comprises a hook-type fabric portion of a hook and loop fastener assembly shaped and sized to cover the port 16 and restrict access therethrough as in FIG. 8. In additional embodiments, the seal 22 may further comprise a tab 36 to allow the wearer to more easily grasp and remove the seal 22, as in FIG. 3.

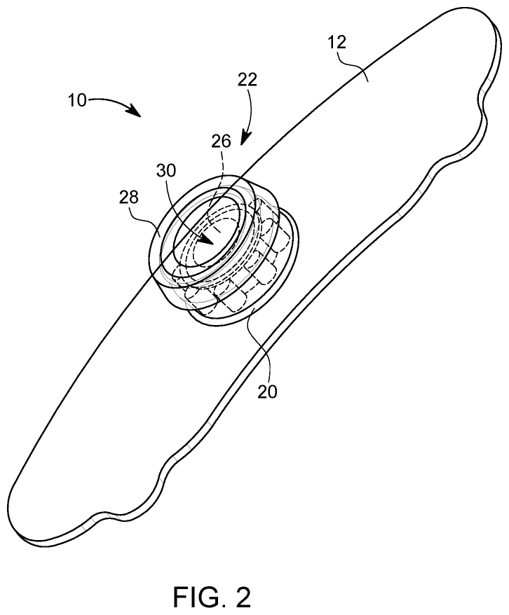

In various embodiments, the seal 22 comprises an enclosure 28 defining a passageway 30 in fluid communication with the port 16, as shown in FIGS. 1, 2, and 6. In additional embodiments, the passageway 30 may be outfitted with a valve 26, such as a one-way valve or other valve configured to restrict flowthrough more in one direction than an opposed direction or to restrict airflow all-together, as in FIGS. 2, 5, and 6.

In various embodiments, the seal 22 further includes an insert 32 that is configured for being received within the passageway 30 to restrict access through the passageway 30 and the port 16, as shown in FIG. 5. In certain embodiments, the insert 32 is configured for interference fitting and may include a tapered shape or similar to provide the interference fitting as in FIG. 5. In further embodiments, the insert 32 includes a valve 26 integrated therein or a filter assembly. In an alternative embodiment, a mesh 34 may be placed within passageway 30 before the insert 32 is interferencingly engaged within passageway 30 as in FIG. 5.

In embodiments, the seal 22 selectively disengages from the mask 10 to allow the wearer to drink through a straw, as shown in FIG. 5. Thereafter, the seal 22 is then selectively re-engaged with the mask 10 and the desired functionality of the mask 10 is then re-established once a wearer is finished.

In particular embodiments, the mask 10 comprises a flexible sheet 12 having a front-facing surface and opposed back-facing surface. In embodiments, the front-facing surface includes a loop-type fabric portion of a hook and loop fastener assembly.

and opposed back-facing surface with adhesive 14 disposed thereupon

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A mouth closure mask assembly comprising:
a flexible sheet having an adhesive disposed on a back facing surface thereof, the adhesive configured for adhering the mask to the face of a wearer;
a port defined though a portion of sheet, the port configured for allowing access between the inside and outside of a wearer's mouth when the port is uncovered;
a retaining flange disposed on the flexible sheet adjacent to the port; and
a seal configured to selectively engage the retaining flange restricting access to the port when the seal and retaining flange are engaged.

2. The mask assembly according to claim 1, wherein the retaining flange is disposed on a portion of a front facing surface of the flexible sheet.

3. The mask assembly according to claim 1, wherein the retaining flange and the seal are magnetically attracted to allow selective engagement.

4. The mask assembly according to claim 3, wherein the seal includes a magnet assembly, the magnet assembly comprising a plurality of magnets mounted within the seal, each of the magnets having a portion configured to magnetically engage the retaining flange and disposed such that each of the plurality of magnets has a polarity that is opposed to that of adjacently disposed magnets mounted within the seal.

5. The mask assembly according to claim 3, wherein the retaining flange includes a magnet assembly, the magnet assembly having a portion configured to magnetically engage the seal.

6. The mask assembly according to claim 3, wherein the seal includes a magnet assembly and the retaining flange includes a ferrous material.

7. The mask assembly according to claim 3, wherein the seal includes a ferrous material and the retaining flange includes a magnet assembly.

8. The mask assembly according to claim 3, wherein the seal and the retaining flange include attractive magnet assemblies.

9. The mask assembly according to claim 1, wherein the seal and the retaining flange are selectively operatively coupled.

10. The mask assembly according to claim 9, wherein the seal and retaining flange are selectively operatively coupled with a hook and loop fastener assembly.

11. The mask assembly according to claim 1, wherein the seal comprises an enclosure defining a passageway in fluid communication with the port.

12. The mask assembly according to claim 11, wherein the seal further comprises one of a one-way valve and a valve that is configured to restrict flowthrough more in one direction than an opposed direction.

13. The mask assembly according to claim 11, wherein the seal further comprises an insert configured to be received within the passageway to restrict access through the passageway and the port.

14. The mask assembly according to claim 11, wherein the insert carries a mesh or filter material.

15. The mask assembly according to claim 1, wherein the seal includes a tab for a wearer to selectively disengage the seal from the retaining flange.

16. A mouth closure mask assembly comprising:
a flexible sheet having a front-facing surface and an opposed back-facing surface;
an adhesive disposed on the back-facing surface of the flexible sheet;
a loop-type fabric portion of a hook and loop fastener assembly, the loop-type fabric portion disposed on the front-facing surface of the flexible sheet;
a port defined though a portion of sheet, the port configured for allowing access between the inside and outside of a wearer's mouth when uncovered; and
a seal comprising a hook-type fabric portion of a hook and loop fastener assembly, the hook-type fabric portion of the seal configured to selectively engage the loop-type fabric portion so the seal covers the port restricting access therethrough.

17. The mask assembly according to claim 16, wherein the seal comprises an enclosure defining a passageway in fluid communication with the port.

18. The mask assembly according to claim 17, wherein the seal further comprises one of a one-way valve and a valve that is configured to restrict flowthrough more in one direction than an opposed direction.

19. The mask assembly according to claim 17, wherein the seal further comprises an insert configured to be received within the passageway to restrict flow through the passageway and the port.

20. The mask assembly according to claim 16, wherein the hook-type fabric portion of the seal fully covers the port preventing access therethrough.
